(19) United States
(12) Patent Application Publication
(10) Pub. No.: US 2023/0159416 A1
(43) Pub. Date: May 25, 2023

(54) DIFLUOROMETHYL IODO COMPOUNDS AND METHODS

(71) Applicant: Recurium IP Holdings, LLC, San Diego, CA (US)

(72) Inventors: Aditya Krishnan Unni, San Diego, CA (US); Joseph Robert Pinchman, San Diego, CA (US); Peter Qinhua Huang, San Diego, CA (US); Kevin Duane Bunker, Escondido, CA (US)

(21) Appl. No.: 17/904,477

(22) PCT Filed: Feb. 17, 2021
(86) PCT No.: PCT/US2021/018392

§ 371 Date: Aug. 17, 2022

Related U.S. Application Data
(60) Provisional application No. 62/979,962, filed on Feb. 21, 2020.

(51) Int. Cl. C07C 17/363; C07C 17/275

(52) U.S. Cl. CPC C07C 17/363; C07C 17/275; C07C 2602/38

(57) ABSTRACT

The present application is related to an improved process for synthesising 1-(difluoromethyl)-3-iodobicyclo[1.1.1]pentane from difluoromethyl iodide and [1.1.1]propellane. Difluoromethyl iodide is made by reacting an iodide salt with chlorodifluoroacetic acid in the presence of a solvent such as sulfolane and an inorganic base, [1.1.1]propellane is synthesised by reacting 1,1-dibromo-2,2-cis(chloromethyl)cyclopropane with a reagent such as magnesium, methyllithium or phenyllithium.

### INCORPORATION BY REFERENCE TO PRIORITY APPLICATION

This application claims priority to U.S. Provisional Application Ser. No. 62/979,962, filed Feb. 21, 2020, which is hereby incorporated herein by reference in its entirety.

### BACKGROUND

### Field

This application relates to processes for making difluoromethyl iodide and 1-(difluoromethyl)-3-iodobicyclo[1.1.1]pentane.

### Description

The compound difluoromethyl iodide (CF₂HI or CHF₂I) is known as an ingredient in compositions useful as refrigerants, solvents, foam blowing agents, and propellants. See, e.g., U.S. Pat. No. 7,083,742. It is traditionally prepared by reacting a difluorocarbene precursor with potassium iodide in the manner disclosed in Cao, P. et. al. J. Chem. Soc., Chem. Commun. 1994, 737-738. Difluoromethyl iodide is also useful as a chemical reagent in organic synthesis. For example, PCT Publication No. WO 2019/139907 discloses a process for making a 0.15M solution of CF₂HI in pentane using the traditional process with a modified workup, then reacting it with tricyclo[1.1.1.0^{1,3}]pentane (also known as [1.1.1]propellane) to produce 1-(difluoromethyl)-3-iodobicyclo[1.1.1]pentane. Improved processes for making CF₂HI are desired, as well as improved methods for making 1-(difluoromethyl)-3-iodobicyclo[1.1.1]pentane.

### SUMMARY

It has now been found that the traditional process for making difluoromethyl iodide using 2,2-difluoro-2-(fluorosulfonyl)acetic acid suffers from inconsistent yields and the use of acetonitrile as solvent can be detrimental to downstream reactions. On larger scales, the traditional process necessitates a tedious work up to remove acetonitrile that produces the difluoromethyl iodide in the form of a relatively dilute solution that complicates further reactions. Although the modified version of the traditional process disclosed in WO 2019/139907 represents an advance in the art, in practice scale up remains complicated by the relatively dilute solutions of CF₂HI produced. An alternative traditional process using chlorodifluoroacetic acid involves the use of stoichiometric amounts of copper (I) iodide (CuI), which are undesirable on large scale due to safety, efficiency and waste management. In addition, it has been reported that only trace amounts of the desired product were obtained when decarboxylation of chlorodifluoroacetic acid was attempted in accordance with the alternative traditional process. See Monfette, S., et al., “Continuous Process for Preparing the Difluoromethylating Reagent [(DMPU)₂Zn(CF₂H)₂] and Improved Synthesis of the ICHF₂ Precursor”, Org. Process Res. Dev., 2020, 24, 6, 1077-1083.

Improved processes have now been developed to address these and other previously unrecognized and/or unappreciated problems. Various embodiments provide a process of making difluoromethyl iodide (CHF₂I), comprising reacting an iodide salt with chlorodifluoroacetic acid under reaction conditions that are selected to produce the difluoromethyl iodide, wherein the reaction conditions include:
an effective amount of a reaction solvent;
an effective amount of the iodide salt dispersed in the reaction solvent; and
an effective amount of an inorganic base dispersed in the reaction solvent.

In an embodiment, at least about 50% by volume of the reaction solvent is sulfolane. In an embodiment, the iodide salt comprises one or both of sodium iodide (NaI) and potassium iodide (KI). In an embodiment, the reaction conditions comprise a reaction temperature in the range of about 40° C. to about 260° C.

Various embodiments provide a process of making difluoromethyl iodide (CHF₂I), comprising reacting an iodide salt with chlorodifluoroacetic acid under reaction conditions that are selected to produce the difluoromethyl iodide, wherein the reaction conditions include:
an effective amount of a reaction solvent, wherein at least about 50% by volume of the reaction solvent is sulfolane;
an effective amount of the iodide salt dispersed in the reaction solvent, wherein the iodide salt comprises one or more of sodium iodide (NaI) and potassium iodide (KI);
an effective amount of an inorganic base dispersed in the reaction solvent; and
a reaction temperature in the range of about 40° C. to about 260° C.

Another embodiment provides a process for making 1-(difluoromethyl)-3-iodobicyclo[1.1.1]pentane, comprising intermixing difluoromethyl iodide with [1.1.1]propellane under reaction conditions that are selected to produce the 1-(difluoromethyl)-3-iodobicyclo[1.1.1]pentane. In an embodiment, the process comprises intermixing an undiluted (neat) difluoromethyl iodide with [1.1.1]propellane under reaction conditions that are selected to produce the 1-(difluoromethyl)-3-iodobicyclo[1.1.1]pentane. In another embodiment, the process comprises intermixing a difluoromethyl iodide solution with [1.1.1]propellane under reaction conditions that are selected to produce the 1-(difluoromethyl)-3-iodobicyclo[1.1.1]pentane. In an embodiment, the concentration of the difluoromethyl iodide in the difluoromethyl iodide solution is in a range of about 0.1M to 10M. In an embodiment, the concentration of the difluoromethyl iodide in the difluoromethyl iodide solution is at least about 0.25M.

These and other embodiments are described in greater detail below.

### DRAWINGS

FIG. 1 illustrates an embodiment of a reactor configuration for making difluoromethyl iodide.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. A group of items linked with the conjunction ‘and’ should not be read as requiring that each and every one of those items be present in the grouping, but rather should be read as ‘and/or’ unless the context indicates otherwise (e.g., in the claims). A group of items linked with the conjunction ‘or’ should not be read as requiring mutual exclusivity among that group, but rather should be read as ‘and/or’ unless the context indicates otherwise. Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Processes of Making Difluoromethyl Iodide

Various embodiments provide a process of making difluoromethyl iodide (CHF₂I), comprising reacting an iodide salt with chlorodifluoroacetic acid under reaction conditions that are selected to produce the difluoromethyl iodide. The reaction conditions comprise an effective amount of a reaction solvent; an effective amount of the iodide salt dispersed in the reaction solvent; and an effective amount of an inorganic base dispersed in the reaction solvent. In various embodiments the reaction conditions further comprise a reaction temperature that is effective to conduct the reaction.

In various embodiments, the reaction solvent utilized in the process is primarily sulfolane. For example, in an embodiment at least about 50% by volume of the reaction solvent is sulfolane. In other embodiments, at least about 80% by volume, at least about 95% by volume or at least about 99% by volume, of the reaction solvent is sulfolane. Sulfolane can be obtained from various commercial sources in a desirably high purity, e.g., about 99% pure. When the reaction solvent is a mixture that contains sulfolane, the balance of the mixture may comprise one or more of various solvents such as DMF, acetonitrile (MeCN), or water. In an embodiment the reaction solvent comprises less than 5% MeCN by volume. Effective amounts of sulfolane-containing reaction solvent may be used to facilitate the course of the reaction and can be determined by routine experimentation informed by the guidance provided herein, including the working examples described below.

In various embodiments, the iodide salt dispersed in the reaction solvent comprises one or more of sodium iodide (NaI) and potassium iodide (KI). In some embodiments, the iodide salt is sodium iodide. In other embodiments, the iodide salt is potassium iodide. The effective amount of the iodide salt dispersed in the reaction solvent is typically selected to be a molar excess based on chlorodifluoroacetic acid. For example, in an embodiment the effective amount of iodide salt is less than a 2× molar excess based on chlorodifluoroacetic acid. In another embodiment the effective amount of iodide salt is less than a 1.5× molar excess based on chlorodifluoroacetic acid. Thus, in various embodiments the effective amount of iodide salt is greater than a molar excess and less than a 1.5× molar excess or less than a 2× molar excess based on chlorodifluoroacetic acid. Effective amounts of iodide salt can be determined by routine experimentation informed by the guidance provided herein, including the working examples described below.

In an embodiment, the use of stoichiometric quantities of copper(I) iodide is reduced or avoided. The use of stoichiometric amounts of transition metal iodide salts may be undesirable when practiced on a large scale due to safety, efficiency and/or waste management considerations. Surprisingly, reaction conditions have now been identified that minimize or render such use of transition metal iodide salts unnecessary. For example, in various embodiments, the iodide salt dispersed in the reaction solvent comprises copper(I) iodide (CuI) in amounts that are less than 10 wt %, less than 9 wt %, less than 8 wt %, less than 7 wt %, less than 6 wt %, less than 5 wt %, less than 4 wt %, less than 3 wt %, less than 2 wt %, less than 1 wt %, or less than 0.5 wt %. The selection of effective amounts of such iodide salts can be determined by routine experimentation informed by the guidance provided herein, including the working examples described below.

In various embodiments the reaction conditions comprise an effective amount of an inorganic base dispersed in the reaction solvent. In an embodiment the inorganic base comprises a potassium cation and a carbonate or phosphate anion. For example, in various embodiments the inorganic base comprises one or more of potassium carbonate (K₂CO₃), potassium bicarbonate (KHCO₃), monopotassium phosphate (KH₂PO₄), dipotassium phosphate (K₂HPO₄), and tripotassium phosphate (K₃PO₄), and/or a hydrated salt of any of the foregoing. In other embodiments the inorganic base comprises a sodium cation and a carbonate or phosphate anion. For example, in various embodiments the inorganic base comprises one or more of sodium carbonate (Na₂CO₃), sodium bicarbonate (NaHCO₃), monosodium phosphate (NaH₂PO₄), disodium phosphate (Na₂HPO₄) and trisodium phosphate (Na₃PO₄), and/or a hydrated salt of any of the foregoing. In an embodiment, the inorganic base comprises potassium carbonate (K₂CO₃), disodium phosphate (Na₂HPO₄) or a mixture thereof, and/or a hydrated salt of any of the foregoing. The effective amount of the inorganic base dispersed in the reaction solvent is typically selected on the basis of the amount of chlorodifluoroacetic acid. For example, in an embodiment the effective amount of inorganic base is an amount that is effective to react with at least about 95 mole % of the chlorodifluoroacetic acid. In another embodiment, the effective amount of inorganic base is an amount that is effective to react with at least about 110 mole % of the chlorodifluoroacetic acid. Those skilled in the art will understand that the equivalent weights of various inorganic bases are generally not the same. Thus, for example, the number of moles of Na₂HPO₄ that is effective to react with at least about 95 mole % of the chlorodifluoroacetic acid is greater than the number of moles of K₃PO₄ because of the difference in valency.

In various embodiments the reaction conditions comprise a reaction temperature that is effective to facilitate the course of the reaction. As used herein, the reaction temperature refers to a temperature of a reaction mixture contained within a reaction vessel, e.g., as measured by a temperature probe in operable contact with the reaction mixture. As noted above, the reaction mixture typically comprises chlorodifluoroacetic acid, an effective amount of a reaction solvent; an effective amount of an iodide salt dispersed in the reaction solvent; and an effective amount of an inorganic base dispersed in the reaction solvent. Various reaction temperatures may be utilized, such as about 40° C. or greater, about 50° C. or greater, about 75° C. or greater, about 100° C. or greater, about 260° C. or less, about 200° C. or less, about 175° C. or less, about 150° C. or less, or any range defined by any two of the foregoing temperatures as endpoints. For example, in various embodiments, the reaction conditions comprise a reaction temperature in a range of from about 40° C. to about 260° C., about 50° C. to about 200° C., about 75° C. to about 175° C., or about 100° C. to about 150° C.

In various embodiments, the reaction conditions, including the amount of chlorodifluoroacetic acid, the amounts and types of reaction solvent, iodide salt (or hydrated salt thereof) and/or reaction temperature, are selected in combination with one another to facilitate the course of the reaction process to produce difluoromethyl iodide. In various embodiments the process is conducted on a relatively large scale, such as on a scale that produces difluoromethyl iodide in an amount per batch of 100 g or more, 1 kg or more, 2 kg or more, or 5 kg or more. Reaction times are typically short and dependent on typical considerations known to those skilled in the art such as reactor volume, temperature, heat transfer and rates at which reactants are added to the reaction mixture. The selection of appropriate reaction conditions for a particular batch can be determined by routine experimentation informed by the guidance provided herein, including the working examples described below. For example, in an embodiment, at least about 95% by volume of the reaction solvent is sulfolane; the iodide salt comprises sodium iodide; the inorganic base comprises potassium carbonate, disodium phosphate or a mixture thereof, or a hydrated salt of any of the foregoing; and the reaction temperature is in the range of about 100° C. to about 150° C.

In various embodiments the difluoromethyl iodide produced by the process is obtained in the form of a difluoromethyl iodide solution having a concentration of at least about 0.25M, at least about 0.5M, at least about 0.75M or at least about 1.0M. The difluoromethyl iodide produced by this process can also be isolated as a neat liquid in a substantially pure state, e.g., at least about 98% or at least about 99% pure. Such undiluted difluoromethyl iodide compositions and difluoromethyl iodide solutions can be used to prepare a number of useful products, such as 1-(difluoromethyl)-3-iodobicyclo[1.1.1]pentane as described below.

### Processes of Making 1-(difluoromethyl)-3-iodobicyclo[1.1.1]pentane

Various embodiments provide a process of making 1-(difluoromethyl)-3-iodobicyclo[1.1.1]pentane, comprising intermixing difluoromethyl iodide with [1.1.1]propellane under reaction conditions that are selected to produce the 1-(difluoromethyl)-3-iodobicyclo[1.1.1]pentane.

The difluoromethyl iodide used in the process may be an undiluted (neat) difluoromethyl iodide or a difluoromethyl iodide solution as described elsewhere herein, or a difluoromethyl iodide composition prepared by another process such as the traditional process or variants thereof described above. In an embodiment, the difluoromethyl iodide is undiluted. As illustrated in Example 12 below, the presence of excessive amounts of acetonitrile can undesirably reduce yields of 1-(difluoromethyl)-3-iodobicyclo[1.1.1]pentane. In various embodiments, the amount of acetonitrile in the difluoromethyl iodide solution is less than 10 wt %, less than 9 wt %, less than 8 wt %, less than 7 wt %, less than 6 wt %, less than 5 wt %, less than 4 wt %, less than 3 wt %, less than 2 wt %, less than 1 wt %, or less than 0.5 wt %. In another embodiment in which a difluoromethyl iodide solution is used, the concentration of the difluoromethyl iodide in the difluoromethyl iodide solution is at least about 0.25M. In another embodiment, the concentration of the difluoromethyl iodide in the difluoromethyl iodide solution is in the range of about 0.1M to about 10M. In an embodiment, a process of making difluoromethyl iodide under the first reaction conditions as described herein further comprises reacting the difluoromethyl iodide with [1.1.1]propellane under second reaction conditions that are selected to produce 1-(difluoromethyl)-3-iodobicyclo[1.1.1]pentane. In various embodiments the second reaction conditions comprise intermixing a difluoromethyl iodide solution with the [1.1.1]propellane, wherein the concentration of the difluoromethyl iodide in the difluoromethyl iodide solution is at least about 0.25M, at least about 0.5M or at least about 1.0M.

The [1.1.1]propellane used in the process may be obtained from various sources or prepared as described herein. For example, in an embodiment, the [1.1.1]propellane is a reaction product of a reaction between 1,1-dibromo-2,2-bis(chloromethyl)cyclopropane and solid magnesium. In another embodiment the [1.1.1]propellane is a reaction product of a reaction between 1,1-dibromo-2,2-bis(chloromethyl)cyclopropane and methyllithium (MeLi). In another embodiment the [1.1.1]propellane is a reaction product of a reaction between 1,1-dibromo-2,2-bis(chloromethyl)cyclopropane and phenyllithium (PhLi).

In an embodiment, the difluoromethyl iodide used in the process of making 1-(difluoromethyl)-3-iodobicyclo[1.1.1]pentane is undiluted and the [1.1.1]propellane is a reaction product of a reaction between 1,1-dibromo-2,2-bis(chloromethyl)cyclopropane and methyllithium or phenyllithium. In an embodiment, the undiluted difluoromethyl iodide is made by a process as described herein.

In an embodiment, the difluoromethyl iodide used in the process of making 1-(difluoromethyl)-3-iodobicyclo[1.1.1]pentane is undiluted and the [1.1.1]propellane is a reaction product of a reaction between 1,1-dibromo-2,2-bis(chloromethyl)cyclopropane and magnesium. In an embodiment, the undiluted difluoromethyl iodide is made by a process as described herein.

In an embodiment, the difluoromethyl iodide used in the process of making 1-(difluoromethyl)-3-iodobicyclo[1.1.1]pentane is a difluoromethyl iodide solution having a concentration of at least about 0.25M (such as at least about 0.5M or at least about 1.0M), and the [1.1.1]propellane is a reaction product of a reaction between 1,1-dibromo-2,2-bis(chloromethyl)cyclopropane and methyllithium or phenyllithium. In an embodiment, the difluoromethyl iodide solution having a concentration of at least about 0.25M (such as at least about 0.5M or at least about 1.0M) is made by a process as described herein.

In an embodiment, the difluoromethyl iodide used in the process of making 1-(difluoromethyl)-3-iodobicyclo[1.1.1]pentane is a difluoromethyl iodide solution having a concentration of at least about 0.25M (such as at least about 0.5M or at least about 1.0M), and the [1.1.1]propellane is a reaction product of a reaction between 1,1-dibromo-2,2-bis(chloromethyl)cyclopropane and magnesium. In an embodiment, the difluoromethyl iodide solution having a concentration of at least about 0.25M (such as at least about 0.5M or at least about 1.0M), is made by a process as described herein.

In various embodiments, the reaction conditions used in the process of making 1-(difluoromethyl)-3-iodobicyclo[1.1.1]pentane are selected in combination with the reaction conditions used to make the difluoromethyl iodide used in the process. In various embodiments one or both of the individual processes are conducted on a relatively large scale, such as on a scale that produces (difluoromethyl)-3-iodobicyclo[1.1.1]pentane in an amount per batch of 100 g or more, 1 kg or more, 2 kg or more, or 5 kg or more. In some embodiments the difluoromethyl iodide is conveniently obtained as a product of a reaction as described herein, and thus the CHF₂I may be undiluted or in the form of a solution of CHF₂I in a nonpolar or polar aprotic solvent, such as a heptane or an MTBE-containing solvent as described elsewhere herein. In certain embodiments, the solution may be made at a CHF₂I concentration of at least about 0.25M (such as at least about 0.5M or at least about 1.0M). Concentrated solutions of CHF₂I can also be obtained by intermixing a nonpolar or polar aprotic solvent with an undiluted CHF₂I or with a more highly concentrated CHF₂I solution. Suitable nonpolar solvents include alkanes such as heptane, and suitable polar aprotic solvents include methyl t-butyl ether (MTBE), diethoxymethane (DEM) and tetrahydrofuran (THF). In some embodiments such relatively highly concentrated CHF₂I solutions can be used directly in a subsequent reaction with [1.1.1]propellane to produce (difluoromethyl)-3-iodobicyclo[1.1.1]pentane. Reaction times for the reaction of difluoromethyl iodide and [1.1.1]propellane are typically short and such reactions may be conducted by intermixing at low reaction temperatures, such as about 35° C. or below. The particular reaction conditions are dependent on typical considerations known to those skilled in the art as informed by the present disclosure, such as reactor volume, temperature, heat transfer and rates at which the difluoromethyl iodide and [1.1.1]propellane are intermixed. The selection of appropriate reaction conditions for a particular batch can be determined by routine experimentation informed by the guidance provided herein, including the working examples described below.

### EXAMPLES

Additional embodiments are disclosed in further detail in the following examples, which are not in any way intended to limit the scope of the claims.

### Example 1

### Preparation of difluoromethyl iodide

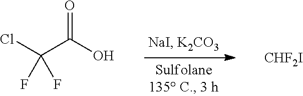

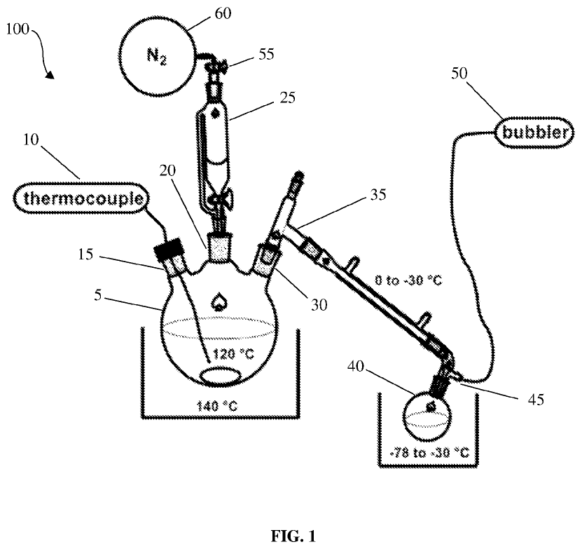

NaI (54.0 g, 360 mmol) and K₂CO₃ (24.9 g, 180 mmol) were added to a 500 mL three-neck flask containing a large stir bar. As illustrated in FIG. 1 by an embodiment of a reactor system 100, the three-neck flask 5 was equipped with a thermocouple 10 in the left port 15 of the flask. The middle port 20 contained a 50 mL addition funnel 25, and the right port 30 had a distillation apparatus 35 with a tared receiving flask 40 cooled to −78° C. The vacuum port 45 on the distillation apparatus 35 was connected to a bubbler 50. The addition funnel 25 had a closed three-way stopcock 55 attached to the top, which allowed the system 100 to be flushed with N₂ from an N₂ source 60 and then closed during the reaction with the only outlet being through the receiving flask 40 and bubbler 50.

Sulfolane (136 mL, 3.5 volumes) was added to the flask and the resulting salt mixture was heated to 150° C. (bath temperature) with an internal (reaction) temperature of 135° C. The slurry was stirred as chlorodifluoroacetic acid (39.1 g, 300 mmol) was added dropwise by the addition funnel with the total addition time being 120 min. As the acid was added, gas evolved continuously from the first addition until the final amount as indicated by the CO₂ flow through the bubbler. The resulting mustard yellow slurry was stirred for an additional hour at an internal (reaction) temperature of 132° C.

The reaction was then cooled to a temperature of 100° C. and 39 mL of MTBE (1 volume) was added to the addition funnel and then added dropwise to the flask over 15 min, carrying the remaining vapor phase product to the receiving flask to provide difluoromethyl iodide (CHF₂I) as an MTBE solution. (At this point, most of the product (>95%) can be distilled prior to the addition of solvent). After the addition of MTBE was complete, the distillation was continued for 5 min. The reaction flask was removed from the heat source and the receiving flask was warmed to 0° C. which allowed CHF₂Cl, a minor side product, to distill out of the receiving flask and escape through the bubbler to a dry ice-acetone trap. The clear, colorless CHF₂I-MTBE solution had a weight of 62.71 g and a density of 1.25 g/mL. ¹H and ¹⁹F NMR spectra indicated the solution was 3.96 M in CHF₂I (198.9 mmol, 66% yield). ¹H NMR (CDCl₃, 400 MHz) δ 7.67 (t, J=56.0 Hz, 1H); ¹⁹F NMR (CDCl₃, 376 MHz) δ −67.3.

### Example 2

### Preparation of difluoromethyl iodide

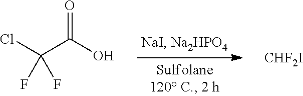

NaI (34 g, 227 mmol) and Na₂HPO₄ (24.9 g, 94 mmol) were added to a 250 mL three-neck flask containing a large stir bar. As illustrated in FIG. 1, the flask was equipped with a thermocouple in the left port of the flask. The middle port contained a 50 mL addition funnel and the right port had a distillation condenser and a tared receiving flask cooled to −78° C. The vacuum port on the distillation apparatus was connected to a bubbler to monitor the flow rate. The addition funnel had a closed three-way stopcock with a N₂ balloon attached to the top, which allowed the system to be flushed with N₂ and then closed during the reaction with the only outlet being through the receiving flask and bubbler.

Sulfolane (64 mL, 4 volumes) was added to the flask and the resulting mixture was heated to 140° C. (bath temperature) with an internal (reaction) temperature of 120° C. The slurry was stirred as chlorodifluoroacetic acid (24.6 g, 189 mmol) was added dropwise from the addition funnel over 60 minutes. As the acid was added, gas evolved continuously from the first addition until the final amount as indicated by the CO₂ flow through the bubbler. The resulting mustard yellow slurry was stirred for an additional 30 min and removed from the heat after no gas was seen escaping the bubbler for at least 10 minutes. At this time, the three-way stopcock connected to the N₂ balloon was opened to allow a gentle stream of N₂ to push any product through the setup into the receiving flask.

The receiving flask was warmed to 0° C. and CHF₂I (25.6 g, 76% yield) was obtained as a clear, colorless liquid.

### Example 3

### Preparation of difluoromethyl iodide

Larger scale example (500 g input of chlorodifluoroacetic acid) using Na₂HPO₄ as a base: NaI (689 g, 4.60 mol) and Na₂HPO₄ (272 g, 1.92 mol) were added to a 3 L three-neck flask containing a large stir bar. The left port of the flask was equipped with a thermometer, the right port was equipped with a 500 mL addition funnel containing the chlorodifluoroacetic acid (500 g, 3.83 mol), and middle right port had a distillation apparatus and tared receiving flasks cooled to −60° C. The vacuum port on the distillation apparatus was connected to a bubbler to monitor the flow rate. The addition funnel had a closed three-way stopcock with a N₂ line attached to the top, which allowed the system to be flushed with N₂ and then closed during the reaction with the only outlet being through the receiving flask and bubbler. Sulfolane (1300 mL) was added to the flask and the resulting mixture was heated to 145° C. (bath temperature) with an internal temperature of 130-135° C. Chlorodifluoroacetic acid was added dropwise from the addition funnel over 2 hr. The reaction was stirred for an additional 2 hr and then cooled after no gas was seen escaping the bubbler for 10 min. At this time, the three-way stopcock connected to the N₂ line was opened to allow a gentle stream of N₂ to push any product remaining through the setup into the receiving flask for 30 min. The receiving flask was warmed to 0° C. and CHF₂I (520 g, 76% yield) was obtained as a clear, colorless liquid.

### Example 4C (Comparative)

### Preparation of difluoromethyl iodide

A modified version of the traditional process (see Cao, P. et. al. J. Chem. Soc., Chem. Commun. 1994, 737-738) was used to prepare CHF₂I as follows: Into a 100 L reactor purged and maintained with an inert atmosphere of nitrogen, was placed MeCN (20 L, 4.0 V), and KI (9.32 Kg, 2.0 eq.). The mixture was stirred and heated to reach an internal temperature at 38±2° C. The temperature was maintained at 40±5° C., and 2,2-difluoro-2-(fluorosulfonyl) acetic acid (5.0 Kg, 1.0 eq.) was added dropwise with stirring, maintaining an internal temperature at 40±5° C. After the addition, the reaction was stirred at 40±5° C. The reaction progress was monitored by ¹H-NMR and ¹⁹F-NMR. Upon completion, the reaction was cooled to −5±5° C., and then the reaction mixture was diluted with ice water (5 V) and heptane (6 V) with stirring for 10 min. The organic phase was separated, and the water phase was extracted with heptane (2 V). The combined organic phases were washed with saturated aqueous NaHCO₃ (5 V×2), cold water (5 V×2). The organic phase was monitored by ¹H-NMR to confirm that MeCN was removed. The organic phase was dried with Na₂SO₄ (0.6 wt %), and then was stirred for 30 min at 0±5° C. after which the stirring was stopped and the drying agent was allowed to settle. The resulting solution of CHF₂I was then transferred into a separate 100 L reactor by peristaltic pump at −5±5° C. in the dark with Na₂SO₄ being retained in the original reactor. The amount of difluoromethyl iodide (CHF₂I) was calculated by qNMR using 3,4,5-trichloropyridine as an internal standard. CHF₂I was obtained as a 0.205 M solution in heptane (40 L, 29% yield).

This example demonstrates the tedious work up that is required to remove acetonitrile from the traditional process and variants and results in a relatively dilute solution of CHF₂I (less than 0.25M).

### Example 5

### Preparation of [1.1.1]propellane (Procedure A)

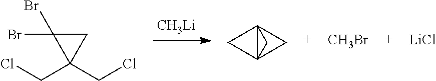

A 3 necked 1 L round bottom flask was charged with methyl lithium (3.1M in DEM (0.807 mol, 260.6 mL) and cooled to −50° C. A solution of 1,1-dibromo-2,2-bis(chloromethyl)cyclopropane (109 g, 0.367 mol in DEM (218 mL) was then added via cannula transfer dropwise over 1 h. After the addition of 1,1-dibromo-2,2-bis(chloromethyl)cyclopropane, the heterogeneous mixture was stirred for 1 h at −50° C. bath before slowly warming to −30° C. The reaction was stirred for an additional 30 min at which point the dry ice bath was replaced with 0° C. ice bath. After 1 h, the reaction flask was placed in a water bath set at 30° C. and connected to a distillation apparatus with the distillate receiving flask immersed in a −50° C. bath. The [1.1.1]propellane was isolated by vacuum distillation (˜100 mbar) and obtained as a 4.7 wt % solution in DEM (423 g, 19.9 g of [1.1.1]propellane, 89% yield). The [1.1.1]propellane solution was stored under nitrogen and used directly in the next step. ¹H NMR (CDCl₃, 400 MHz) δ 1.93 (s, 6H).

### Example 6

### Preparation of [1.1.1]propellane (Procedure B)

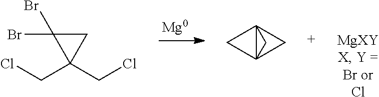

Magnesium turnings (7.29 g, 300 mmol) were added to an oven dried 500 mL single neck flask containing a stir bar. The flask was fitted with a rubber septum with a digital thermocouple penetrating it such that the tip of the thermocouple was at the base of the flask. The flask was evacuated and backfilled with nitrogen while still hot. After reaching rt, 50 mL of anhydrous THF was added to the flask followed by the dropwise addition of 10 mL of a 1.0M solution of diisobutylaluminum hydride in THF. The magnesium turnings were stirred in the flask for 1 hour to fully activate the turnings. After 1 h, an additional 30 mL of THF was added to the flask containing the Mg turnings. The flask was immersed in a water bath maintained at rt to moderate the reaction temperature. 1,1-Dibromo-2,2-bis(chloromethyl)cyclopropane (30 g, 101 mmol) was added to a separate 100 mL conical flask and dissolved in THF (90 mL). The solution of 1,1-dibromo-2,2-bis(chloromethyl)cyclopropane was added to the magnesium turnings via cannula dropwise over 60 min, while maintaining the temperature of the reaction between 20-35° C. After the addition was complete, the reaction was stirred for an additional hour at ambient temperature. The content of [1.1.1]propellane in the resulting THF solution was determined using ¹H NMR using p-xylenes as an internal standard. The ratio of integration indicated a 55% yield of [1.1.1]propellane.

The crude [1.1.1]propellane solution was connected to a distillation apparatus and placed in a water bath set at 30° C. and the product was vacuum distilled. The distillate receiving flask was immersed in a −78° C. bath. The [1.1.1]propellane solution was distilled over as a solution in THF and used directly in the next step.

### Example 7

### Preparation of [1.1.1]propellane (Procedure C)

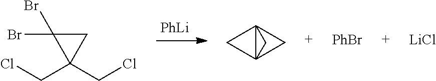

To a stirred solution of 1,1-dibromo-2,2-bis(chloromethyl)cyclopropane (25 g, 84.23 mmol) in Et₂O (80 mL) at −45° C. was added phenyl lithium (1.9 M in dibutyl ether, 89.2 mL, 168.5 mmol) dropwise over 15 min. The reaction mixture was stirred at −45° C. for 30 min and then the cooling bath was removed and replaced with an ice bath. After 2 h, the product was distilled by vacuum distillation at 0-5° C. with the distillate collected at −78° C. to obtain [1.1.1]propellane (0.53 M in Et₂O, 52 mL, 32% yield) as a colorless solution. The [1.1.1]propellane solution was stored under nitrogen and used directly in the next step.

### Example 8

### Preparation of 1-(difluoromethyl)-3-iodobicyclo[1.1.1]pentane

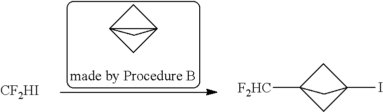 [SMILES: C1C23CC12C3 ; "CF2HI" ; FC(F)C12CC(I)(C1)C2]

This process utilized a difluoromethyl iodide solution prepared as described in Example 1 and a [1.1.1]propellane solution prepared as described in Example 6 (Procedure B). A dry, 100 mL, single-neck round-bottom flask containing a stir bar was fitted with a septum and flushed with a nitrogen filled balloon. The vessel was cooled to 0° C. before reagent grade diethoxymethane (DEM) (20.1 mL) and difluoromethyl iodide (5.3 M in MTBE, 3.1 mL, 16.5 mmol) were added sequentially. While stirring, [1.1.1]propellane (0.37 M in THF, 40.3 mL, 15.0 mmol) was added over 5 min. The reaction was then removed from the ice bath and allowed to warm to room temperature.

After 24 hours at room temperature, analysis of the ¹H NMR spectrum indicated full conversion of [1.1.1]propellane to the desired product. The clear colorless solution was concentrated in vacuo, keeping the bath temperature at 20° C. After the solvent was removed, the vessel was dried under vacuum at room temperature to afford 1-(difluoromethyl)-3-iodobicyclo[1.1.1]pentane (2.70 g, ˜93 wt % (contained residual THF), 10.4 mmol, 69% yield) as a white solid. ¹H NMR (CDCl3, 400 MHz): 5.64 (t, J=56.0 Hz, 1H), 2.39 (s, 6H).

### Example 9

### Preparation of 1-(difluoromethyl)-3-iodobicyclo[1.1.1]pentane

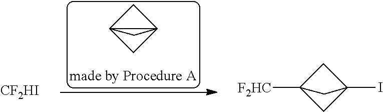 [SMILES: C1C23CC12C3 ; "CF2HI" ; FC(F)C12CC(I)(C1)C2]

This process utilized a solution of difluoromethyl iodide in diethoxymethane and a [1.1.1]propellane solution prepared using the procedure described in Example 5 (Procedure A). To a solution of [1.1.1]propellane (4.09 wt % in DEM, 22 kg, 13.61 mol) was added difluoromethyl iodide (2.91 kg, 16.34 mol) in DEM (6 L) at 0-5° C. The mixture was warmed to 25° C. and stirred at 25-30° C. for 48 h. The mixture was then concentrated to afford 1-(difluoromethyl)-3-iodobicyclo[1.1.1]pentane (2.31 kg, 70% yield) as an off-white solid.

### Example 10

### Preparation of 1-(difluoromethyl)-3-iodobicyclo[1.1.1]pentane

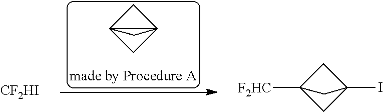 [SMILES: C1C23CC12C3 ; "CF2HI" ; FC(F)C12CC(I)(C1)C2]

This process utilized a solution of difluoromethyl iodide in heptane and a [1.1.1]propellane solution prepared as described in Example 5 (Procedure A). To a 3 neck round bottomed flask cooled to 0° C. was added [1.1.1]propellane (5.9 wt % in DEM, 84.7 g, 0.076 mol) followed by a pre-cooled (0° C.) solution of CF₂HI (18 wt % in heptane, 100 g, 0.101 mol) in one portion. After the mixture was stirred for 10 min at 0° C., the cooling bath was removed, and the reaction was stirred at 25° C. for 2 h. The reaction was concentrated in vacuo to afford 1-(difluoromethyl)-3-iodobicyclo[1.1.1]pentane as a white solid (17.8 g, 91% yield).

### Example 11

### Preparation of 1-(difluoromethyl)-3-iodobicyclo[1.1.1]pentane

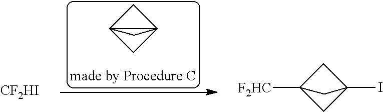 [SMILES: C1C23CC12C3 ; "CF2HI" ; FC(F)C12CC(I)(C1)C2]

This process utilized a solution of difluoromethyl iodide in pentane and a [1.1.1]propellane solution prepared as described in Example 7 (Procedure C). To a stirred solution of [1.1.1]propellane (0.53 M in diethyl ether, 52 mL, 27.6 mmol) at −40° C. was added CF₂HI (0.15 M in pentane, 200 mL, 30 mmol). The reaction mixture was warmed to room temperature and stirred for 2 days. The reaction mixture was then concentrated in vacuo at 0-5° C. to afford 1-(difluoromethyl)-3-iodobicyclo[1.1.1]pentane (5 g, 75% yield) as a solid.

### Example 12

### Preparation of 1-(difluoromethyl)-3-iodobicyclo[1.1.1]pentane

CHF₂I was prepared following the previously published process (see Cao, P. et. al. J. Chem. Soc., Chem. Commun. 1994, 737-738) as follows: A round bottom flask purged and maintained with an atmosphere of nitrogen, and MeCN (500 mL), and KI (186 g, 1.12 mol) were added. The mixture was stirred and heated to reach an internal temperature at 40° C. The temperature was maintained at 40° C. and 2,2-difluoro-2-(fluorosulfonyl) acetic acid (106 g, 0.596 mol) in MeCN (40 mL), was added dropwise with stirring, maintaining an internal temperature at 40° C. After the addition, the reaction was stirred at 60° C. for 2 h. Upon completion, the product was distilled to provide CHF₂I (32 wt % in acetonitrile, 50 g, 15% yield).

Step 2: To a solution of [1.1.1]propellane (0.23 M in Et₂O, 100 mL, 23 mmol) at −40° C. was added CHF₂I (32 wt % in acetonitrile, 25 g, 45 mmol). The reaction mixture was warmed and stirred at rt. After 48 h, no product was observed by ¹H NMR analysis. This example illustrates the detrimental effect of excessive acetonitrile on yield.

1. A process of making difluoromethyl iodide (CHF₂I), comprising reacting an iodide salt with chlorodifluoroacetic acid under reaction conditions that are selected to produce the difluoromethyl iodide, wherein the reaction conditions comprise:
an effective amount of a reaction solvent;
an effective amount of the iodide salt dispersed in the reaction solvent; and
an effective amount of an inorganic base dispersed in the reaction solvent.

2. The process of claim 1, wherein at least about 50% by volume of the reaction solvent is sulfolane.

3. The process of claim 2, wherein at least about 80% by volume of the reaction solvent is sulfolane.

4. The process of claim 3, wherein at least about 95% by volume of the reaction solvent is sulfolane.

5. The process of claim 1, wherein the iodide salt comprises one or more of sodium iodide (NaI) and potassium iodide (KI).

6. The process of claim 1, wherein the reaction conditions comprise a reaction temperature in the range of about 40° C. to about 260° C.

7. The process of claim 1, wherein the reaction temperature is in the range of about 50° C. to about 200° C.

8. The process of claim 1, wherein the reaction temperature is in the range of about 75° C. to about 175° C.

9. The process of claim 1, wherein the reaction temperature is in the range of about 100° C. to about 150° C.

10. The process of claim 1, wherein the effective amount of inorganic base is an amount that is effective to react with at least about 95 mole % of the chlorodifluoroacetic acid.

11. The process of claim 1, wherein the effective amount of inorganic base is an amount that is effective to react with at least about 110 mole % of the chlorodifluoroacetic acid.

12. The process of claim 1, wherein the inorganic base comprises one or more of potassium carbonate (K₂CO₃), potassium bicarbonate (KHCO₃), monopotassium phosphate (KH₂PO₄), dipotassium phosphate (K₂HPO₄), tripotassium phosphate (K₃PO₄), sodium carbonate (Na₂CO₃), sodium bicarbonate (NaHCO₃), monosodium phosphate (NaH₂PO₄), disodium phosphate (Na₂HPO₄) and trisodium phosphate (Na₃PO₄), or a hydrated salt of any of the foregoing.

13. The process of claim 1, wherein the inorganic base comprises potassium carbonate (K₂CO₃), disodium phosphate (Na₂HPO₄) or a mixture thereof, or a hydrated salt of any of the foregoing.

14. The process of claim 1, wherein the effective amount of iodide salt is less than a 2× molar excess based on chlorodifluoroacetic acid.

15. The process of claim 1, wherein the effective amount of iodide salt is less than a 1.5× molar excess based on chlorodifluoroacetic acid.

16. The process of claim 1, wherein the iodide salt comprises less than 10 wt % copper(I) iodide (CuI).

17. The process of claim 1, wherein:
at least about 95% by volume of the reaction solvent is sulfolane;
the iodide salt comprises sodium iodide (NaI);
the inorganic base comprises potassium carbonate (K₂CO₃), disodium phosphate (Na₂HPO₄) or a mixture thereof, or a hydrated salt of any of the foregoing; and
the reaction conditions comprise a reaction temperature in the range of about 100° C. to about 150° C.

18. The process of claim 1, further comprising reacting the difluoromethyl iodide with [1.1.1]propellane under second reaction conditions that are selected to produce 1-(difluoromethyl)-3-iodobicyclo[1.1.1]pentane.

19. The process of claim 18, wherein the second reaction conditions comprise intermixing a difluoromethyl iodide solution with the [1.1.1]propellane, wherein the concentration of the difluoromethyl iodide in the difluoromethyl iodide solution is at least about 0.5M.

20. The process of claim 18, wherein the [1.1.1]propellane is a reaction product of a reaction between 1,1-dibromo-2,2-bis(chloromethyl)cyclopropane and solid magnesium.

21. The process of claim 18, wherein the [1.1.1]propellane is a reaction product of a reaction between 1,1-dibromo-2,2-bis(chloromethyl)cyclopropane and methyllithium (MeLi).

22. The process of claim 18, wherein the [1.1.1]propellane is a reaction product of a reaction between 1,1-dibromo-2,2-bis(chloromethyl)cyclopropane and phenyllithium (PhLi).

23. A process for making 1-(difluoromethyl)-3-iodobicyclo[1.1.1]pentane, comprising intermixing difluoromethyl iodide (CHF₂I) with [1.1.1]propellane under reaction conditions that are selected to produce the 1-(difluoromethyl)-3-iodobicyclo[1.1.1]pentane.

24-32. (canceled)
